(19) United States
(12) Patent Application Publication
(10) Pub. No.: US 2023/0420115 A1
(43) Pub. Date: Dec. 28, 2023

(54) MEDICAL CARE ASSISTANCE SYSTEM AND INPUT ASSISTANCE METHOD FOR MEDICAL CARE INFORMATION

(71) Applicant: OLYMPUS MEDICAL SYSTEMS CORP., Tokyo (JP)

(72) Inventors: Tatsuya HONOKI, Tokyo (JP); Kazuyoshi TAMURA, Tokyo (JP); Haruhiko SAKAYORI, Tokyo (JP)

(73) Assignee: OLYMPUS MEDICAL SYSTEMS CORP., Tokyo (JP)

(21) Appl. No.: 18/367,610

(22) Filed: Sep. 13, 2023

Related U.S. Application Data
(63) Continuation of application No. PCT/JP2021/010350, filed on Mar. 15, 2021.

(51) Int. Cl. G16H 30/40; G16H 30/20

(52) U.S. Cl. CPC G16H 30/40; G16H 30/20

(57) ABSTRACT

An image acquisition unit acquires a medical image. An item recording unit records a plurality of items representing options for examination results. An item identifying unit identifies a display item group including one or more items from a plurality of items that can be included in an input screen. A display screen generation unit displays an input screen that is for a user to enter an examination result on the medical image and in which one or more items included in the display item group are arranged. An operation reception unit that receives a selection operation for a displayed item.

### CROSS-REFERENCE TO RELATED APPLICATIONS

This application is based upon and claims the benefit of priority from the International Application No. PCT/JP2021/010350, filed on Mar. 15, 2021, the entire contents of which is incorporated herein by reference.

### BACKGROUND

### 1. Field of the Invention

The present disclosure relates to a medical care assistance system and an input assistance method for medical care information.

### 2. Description of the Related Art

An endoscopic observation device is connected to an endoscope inserted into the digestive tract of a patient and displays an image of the inside of the digestive tract being imaged by the endoscope on the display device in real time. The doctor operates a release switch of the endoscope, and the endoscopic observation device captures an endoscopic image when the release switch is operated, and transmits the captured endoscopic image to an image accumulation server.

After the completion of the endoscopic examination, the doctor operates an information processor such as a personal computer so as to read endoscopic images captured during the examination from the image accumulation server and display the images on the display device in order to prepare an examination report. The doctor selects endoscopic images that include an abnormal site such as a lesion, attaches the images to the examination report, and enters examination results related to the attached endoscopic images on a report input screen.

Japanese Patent Application Publication No. 2018-194970 discloses a report input screen including an examination result input area for doctors to enter examination results. The report input screen disclosed in Japanese Patent Application Publication No. 2018-194970 displays options for examination results and includes a user interface that allows the doctor to enter examination results by selecting a check box.

### SUMMARY

Tens to hundreds of items are included as options for examination results on the report input screen, and the items serving as options therefore do not fit on one page and are displayed on multiple pages. In order to select the item to be recorded, the user needs to display a page including the item and further search for the item from the page, and the large number of display items is a factor that increases the report creation time. In this background, a general purpose of the present disclosure is to provide technology for enabling efficient input of examination results by the user.

A medical care assistance system according to an embodiment of the present disclosure includes: an acquisition unit that acquires a medical image; an item recording unit that records a plurality of items representing options for examination results; an item identifying unit that identifies a display item group including one or more items from a plurality of items that can be included in an input screen; a display screen generation unit that displays an input screen that is for a user to enter an examination result on the medical image and in which one or more items included in the display item group are arranged; and an operation reception unit that receives a selection operation for a displayed item.

A medical care assistance system according to another embodiment of the present disclosure includes: an image-capturing unit that captured a medical image; an acquisition unit that acquires a medical image; an item recording unit that records a plurality of items representing options for examination results; an item identifying unit that identifies a display item group including one or more items from a plurality of items that can be included in an input screen; a display screen generation unit that displays an input screen that is for a user to enter an examination result on the medical image and in which one or more items included in the display item group are arranged; and an operation reception unit that receives a selection operation for a displayed item.

A medical care assistance method according to another embodiment of the present disclosure includes: acquiring a medical image; identifying a display item group including one or more items from a plurality of items that can be included in an input screen; displaying an input screen that is for a user to enter an examination result on the medical image and in which one or more items included in the display item group are arranged; and receiving a selection operation for a displayed item.

Optional combinations of the aforementioned constituting elements and implementations of the present disclosure in the form of methods, apparatuses, systems, recording mediums, and computer programs may also be practiced as additional modes of the present disclosure.

### BRIEF DESCRIPTION OF THE DRAWINGS

Embodiments will now be described, by way of example only, with reference to the accompanying drawings that are meant to be exemplary, not limiting, and wherein like elements are numbered alike in several figures, in which:

FIG. 1 is a diagram showing the configuration of a medical care assistance system;

FIG. 2 is a diagram showing an example of a list screen for endoscopic images;

FIG. 3 is a diagram showing an example of a report input screen;

FIG. 4 is a diagram showing examples of additional information;

FIG. 5 is a diagram showing an example of the report input screen;

FIG. 6 is a diagram showing another example of the report input screen;

FIG. 7 is a diagram showing examples of past examination results of a plurality of patients;

FIG. 8 is a diagram showing an example of a search result for examination results that satisfy display conditions;

FIG. 9 is a diagram showing another example of the report input screen;

FIGS. 10A and 10B are figures showing an example of a check mark;

FIG. 11 is a diagram showing an example of past examination results of a patient;

FIG. 12 is a diagram showing another example of the report input screen;

FIG. 13 is a diagram showing another example of the report input screen;

FIG. 14 is a diagram showing an example of medical procedure implementation information;

FIG. 15 is a diagram showing a table for converting implementation items into items in a medical care assistance system;

FIG. 16 is a diagram showing another example of the report input screen;

FIG. 17 is a diagram showing a display example of an input content display area; and

FIG. 18 is a diagram showing a display example of an input content display area.

### DETAILED DESCRIPTION

The disclosure will now be described by reference to the preferred embodiments. This does not intend to limit the scope of the present disclosure, but to exemplify the disclosure.

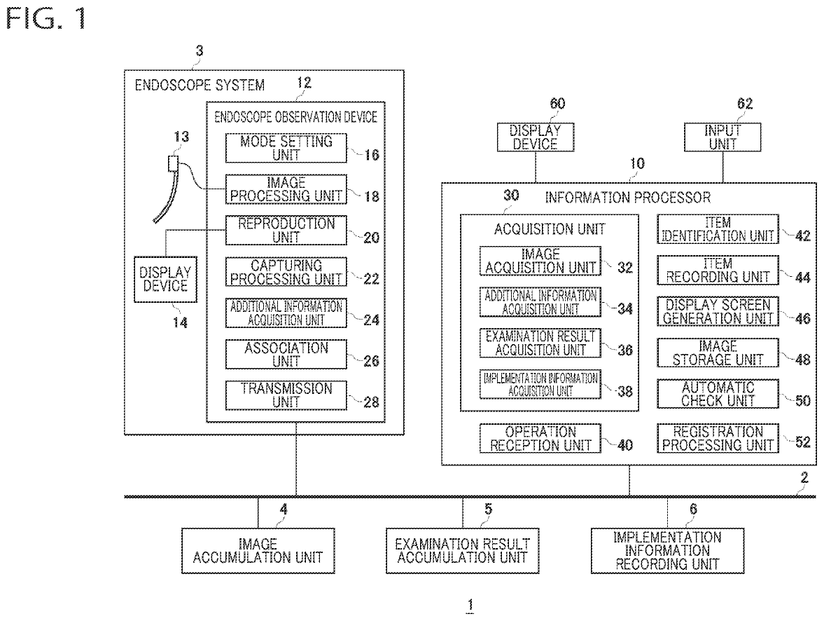

FIG. 1 shows the configuration of a medical care assistance system 1 according to an embodiment. The medical care assistance system 1, which is a medical support system, is provided in a medical facility such as a hospital where endoscopic examinations are performed. In the medical care assistance system 1, an endoscope system 3, an image accumulation unit 4, an examination result accumulation unit 5, an implementation information recording unit 6, and an information processor 10 are connected communicatively via a network 2 such as a local area network (LAN). The image accumulation unit 4, the examination result accumulation unit 5, and the implementation information recording unit 6 may each be configured as a recording server.

The endoscope system 3 is provided in an examination room, includes an endoscopic observation device 12, an endoscope 13, and a display device 14, and has a function of generating an endoscopic image in a plurality of observation modes. The endoscopic observation device 12 includes a mode setting unit 16, an image processing unit 18, a reproduction unit 20, a capturing processing unit 22, an additional information acquisition unit 24, an association unit 26, and a transmission unit 28.

The configuration of the endoscopic observation device 12 is implemented by hardware such as an arbitrary processor, memory, auxiliary storage, or other LSIs and by software such as a program or the like loaded into the memory. The figure depicts functional blocks implemented by the cooperation of hardware and software. Thus, a person skilled in the art should appreciate that there are many ways of accomplishing these functional blocks in various forms in accordance with the components of hardware only, software only, or the combination of both.

The endoscope 13 has a light guide for illuminating the inside of a subject by transmitting illumination light supplied from the endoscopic observation device 12, and the distal end of the endoscope 13 is provided with an illumination window for emitting the illumination light transmitted by the light guide to the subject and an image-capturing unit for image-capturing the subject at a predetermined cycle and outputting an image-capturing signal to the endoscopic observation device 12. The endoscopic observation device 12 supplies illumination light according to the observation mode to the endoscope 13. The image-capturing unit includes a solid-state imaging device, e.g., a CCD image sensor or a CMOS image sensor, that converts incident light into an electric signal.

The image processing unit 18 performs image processing on the image-capturing signal photoelectrically converted by a solid-state imaging device of the endoscope 13 so as to generate an endoscopic image, and the reproduction unit 20 displays the endoscopic image on the display device 14 in real time. In addition to normal image processing such as A/D conversion and noise removal, the image processing unit 18 includes a function of performing special image processing for the purpose of highlighting. Being equipped with a special image processing function, the image processing unit 18 allows the endoscopic observation device 12 to generate an endoscopic image that has not undergone special image processing and an endoscopic image that has undergone the special image processing from an image-capturing signal resulting from image capturing using the same illumination light.

The mode setting unit 16 sets the observation mode according to an instruction from the doctor. The observation mode is determined by the combination of the image-capturing method, illumination method, of the subject and the image processing method of the image-capturing signal. The endoscope system 3 may have the following observation modes.

White Light Imaging (WLI) Observation Mode

The WLI observation mode is an observation mode where the endoscope 13 irradiates the subject with normal light, white light, so as to capture an image of the subject and where the image processing unit 18 performs normal image processing such as noise reduction on the image-capturing signal so as to generate an endoscopic image.

Texture and Color Enhancement Imaging (TXI) Observation Mode

The TXI observation mode is an observation mode where the endoscope 13 irradiates the subject with normal light, which is white light, so as to capture an image of the subject and where the image processing unit 18 performs special image processing that optimizes three elements of “structure”, “color tone”, and “brightness” of a mucosal surface after performing normal image processing such as noise reduction on the image-capturing signal so as to generate an endoscopic image.

Red Dichromatic Imaging (RDI) Observation Mode

The RDI observation mode is an observation mode where the endoscope 13 irradiates the subject with green, amber, and red narrow band light so as to capture an image of the subject and where the image processing unit 18 performs normal image processing such as noise reduction on the image-capturing signal so as to generate an endoscopic image.

Narrow Band Imaging (NBI) Observation Mode

The NBI observation mode is an observation mode where the endoscope 13 irradiates the subject with blue and green narrow band light so as to capture an image of the subject and where the image processing unit 18 performs normal image processing such as noise reduction on the image-capturing signal so as to generate an endoscopic image.

Autofluorescence Imaging (AFI) Observation Mode

The AFI observation mode is an observation mode where the endoscope 13 irradiates the subject with excitation light, in the range of 390-470 nm, so as to capture an image of the subject and where the image processing unit 18 performs normal image processing such as noise reduction on the image-capturing signal and then generates an endoscopic image converted to green according to the signal strength.

The doctor selects an observation mode suitable for the observation situation and displays the endoscopic image on the display device 14. When the doctor operates the release switch of the endoscope 13, the capturing processing unit 22 captures (saves) an endoscopic image generated by the image processing unit 18 at the time when the release switch is operated. At this time, the additional information acquisition unit 24 acquires information indicating the observation mode of the endoscopic image, hereinafter simply referred to as “observation mode information”, from the mode setting unit 16, and the association unit 26 associates the observation mode information with the captured endoscopic image as additional information. The association unit 26 may add observation mode information as metadata to the captured endoscopic image.

The endoscope system 3 according to an embodiment has an image analysis function of deriving information indicating a site of the subject included in the endoscopic image, information on a lesion, and information on endoscopic treatment from the endoscopic image. This image analysis function may be realized using a trained model generated by machine learning of endoscopic images captured in the past. When the additional information acquisition unit 24 inputs a captured endoscopic image to the trained model, the trained model outputs information indicating a site of the subject included in the endoscopic image, information on a lesion, and information on endoscopic treatment.

Information indicating a site of the subject, hereinafter also referred to simply as “site information”, may include the organ name and site name of the image-captured subject or may include either the organ name or the site name. Information on a lesion, hereinafter also referred to simply as “lesion information”, may include information indicating whether the lesion is being image-captured in the endoscopic image and may include information indicating the type of the lesion if the lesion is being image-captured. The information on endoscopic treatment, hereinafter also referred to simply as “treatment information”, indicates whether the endoscopic image contains traces of treatment performed by an endoscopic treatment tool, that is, information indicating whether treatment performed by an endoscopic treatment tool has been performed. If treatment has been performed, the information on endoscopic treatment may contain information indicating the type of the treatment.

When the additional information acquisition unit 24 acquires site information, lesion information, and treatment information from the trained model, the association unit 26 associates the site information, the lesion information, and the treatment information with the captured endoscopic image as additional information. The association unit 26 may add the site information, the lesion information, and the treatment information as metadata to the captured endoscopic image. The additional information acquisition unit 24 may acquire the site information, the lesion information, and the treatment information through means other than the trained model. In the embodiment, the association unit 26 associates all of the observation mode information, the site information, the lesion information, and the treatment information with the endoscopic image as additional information. Alternatively, in another example, at least one of the observation mode information, the site information, the lesion information, and the treatment information may be associated with the endoscopic image as additional information.

The transmission unit 28 transmits the endoscopic image associated with the additional information to the image accumulation unit 4. Every time the capturing processing unit 22 captures an endoscopic image, the transmission unit 28 may transmit the captured endoscopic image to the image accumulation unit 4. Alternatively, the transmission unit 28 may transmit endoscopic images captured during an examination to the image accumulation unit 4 all at once after the examination is completed.

The image accumulation unit 4 records a plurality of endoscopic images transmitted from the endoscopic observation device 12 in association with an examination ID for identifying the endoscopic examination. When the image accumulation unit 4 receives a request to read an endoscopic image with a specified examination ID from the information processor 10, the image accumulation unit 4 transmits a plurality of endoscopic images associated with the examination ID to the information processor 10.

The information processor 10 is installed in a room other than the examination room and is used by a doctor to prepare an examination report. The input unit 62 is a tool for the user to input operations, such as a mouse, a stylus, or a keyboard. The information processor 10 includes an acquisition unit 30, an operation reception unit 40, an item identification unit 42, an item recording unit 44, a display screen generation unit 46, an image storage unit 48, an automatic check unit 50, and a registration processing unit 52. The acquisition unit 30 has an image acquisition unit 32, an additional information acquisition unit 34, an examination result acquisition unit 36, and an implementation information acquisition unit 38.

The item recording unit 44 records a plurality of items representing options for examination results for all observed organs and all diagnostic items. In the embodiment, the item recording unit 44 is shown as a component of the information processor 10. However, the item recording unit 44 may be managed by a management server or the like at the medical facility.

The information processor 10 shown in FIG. 1 includes a computer. Various functions shown in FIG. 1 are realized by the computer executing a program. The computer includes a memory for loading programs, one or more processors that execute loaded programs, auxiliary storage, and other LSIs as hardware. The processor may be formed with a plurality of electronic circuits including a semiconductor integrated circuit and an LSI, and the plurality of electronic circuits may be mounted on one chip or on a plurality of chips. The functional blocks shown in FIG. 1 are realized by cooperation between hardware and software. Therefore, a person skilled in the art should appreciate that there are many ways of accomplishing these functional blocks in various forms in accordance with the components of hardware only, software only, or the combination of both.

After the completion of an endoscopic examination, the user, a doctor, enters a user ID and a password to the information processor 10 so as to log in. An application for preparing an examination report is started when the user logs in, and a list of already performed examinations is displayed on the display device 60. The list of already performed examinations displays examination information such as the patient name, the patient ID, the examination date and time, the examination type, and the like in a list, and the user operates the input unit 62 so as to select an examination for which a report is to be prepared. When the operation reception unit 40 receives an examination selection operation, the image acquisition unit 32 acquires a plurality of endoscopic images linked to the examination ID of the selected examination from the image accumulation unit 4 and stores the endoscopic images in the image storage unit 48, and the display screen generation unit 46 generates a list screen for the endoscopic images and displays the endoscopic images on the display device 60.

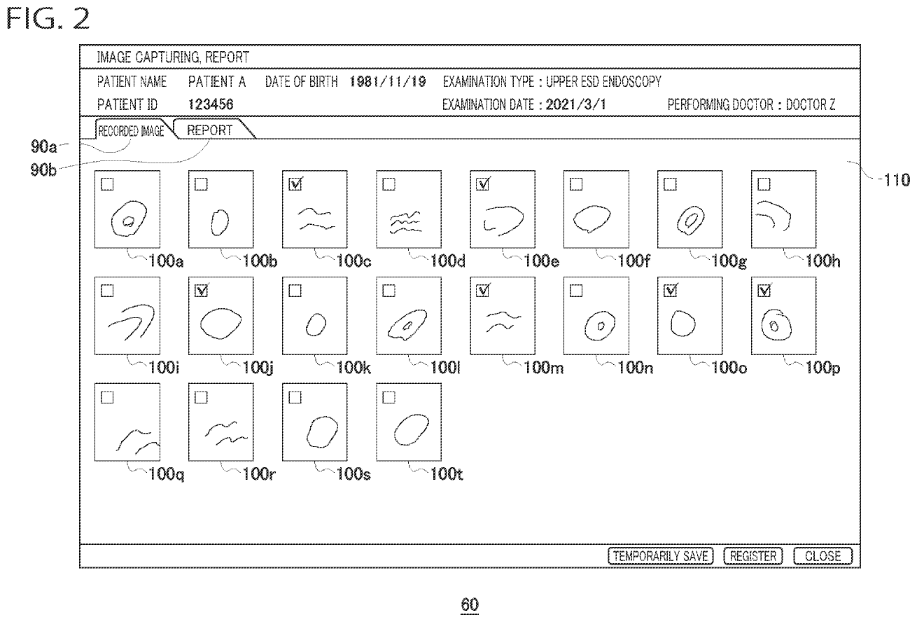

FIG. 2 shows an example of a list screen for endoscopic images. The display screen generation unit 46 displays endoscopic images 100a to 100t, hereinafter referred to as “endoscopic images 100” unless otherwise distinguished, acquired by the image acquisition unit 32 in an image display area 110 according to the order of the capturing of the endoscopic images. The list screen for the endoscopic images is displayed on the display device 60 while a temporarily save tab 90a is being selected. In the upper part of the list screen, information such as a patient name, a patient ID, the date of birth, an examination type, an examination date, and a performing doctor is displayed. These pieces of information are contained in examination order information and may be acquired from the management server of the medical facility.

Each endoscopic image 100 is provided with a check box, and the endoscopic image 100 is selected as an attached image of the report when the user operates a mouse and places a mouse pointer on the check box and right-clicks. The endoscopic image 100 can be enlarged when the user places the mouse pointer on the endoscopic image 100 and right-clicks, and the user may determine whether to attach the endoscopic image to the report while looking at the enlarged endoscopic image.

In the example shown in FIG. 2, check marks are displayed in the check boxes of the endoscopic images 100c, 100e, 100j, 100m, 100o, and 100p that indicate that the check boxes are being selected. The registration processing unit 52 temporarily registers the selected endoscopic images 100c, 100e, 100j, 100m, 100o, and 100p in the image storage unit 48 as report attached images when the user operates a temporarily save button using the input unit 62. After selecting the attached images, the user selects a report tab 90b to display a report input screen on the display device 60.

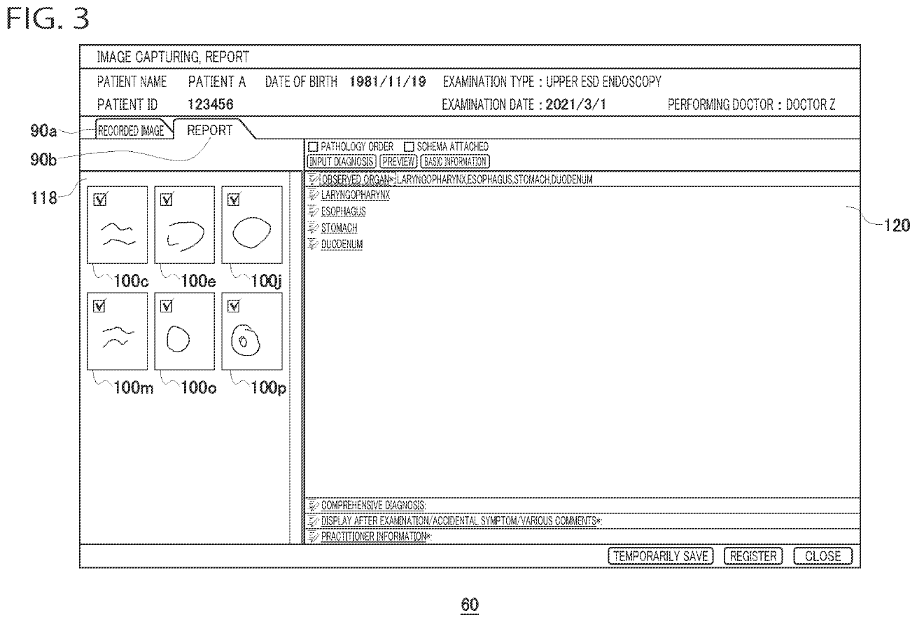

FIG. 3 illustrates an example of the report input screen. Upon the selection of the report tab 90b, the display screen generation unit 46 generates a report input screen for the user to input examination results related to the endoscopic images and displays the report input screen on the display device 60. The report input screen includes two areas: an attached image display area 118 for displaying attached images on the left side; and an input area 120 for the user to input the examination results on the right side. In this example, the endoscopic images 100c, 100e, 100j, 100m, 100o, and 100p are selected as attached images and displayed in the attached image display area 118.

Once an endoscopic image 100 is provisionally registered as a report attached image, the additional information acquisition unit 34 acquires additional information associated with the endoscopic image 100. In the embodiment, the additional information acquisition unit 34 acquires the additional information from the endoscopic image stored in the image storage unit 48. Alternatively, the additional information acquisition unit 34 may acquire additional information from the image accumulation unit 4.

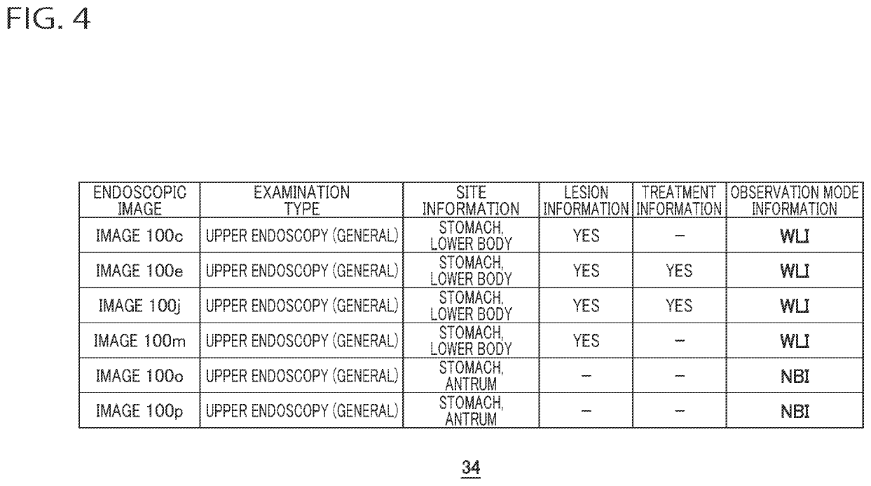

FIG. 4 shows examples of additional information acquired by the additional information acquisition unit 34. The additional information acquisition unit 34 acquires site information, lesion information, treatment information, and observation mode information as additional information contained in the report attached image. FIG. 4 shows additional information of the endoscopic images 100c, 100e, 100j, 100m, 100o, and 100p selected as attached images. The lesion information shown in FIG. 3 is information indicating whether a lesion is present. However, the lesion information may further include information indicating the type of lesion when a lesion is present. In the same way, although the treatment information is information indicating whether endoscopic treatment has been performed, the treatment information may further include information indicating the type of endoscopic treatment when endoscopic treatment has been performed. In the embodiment, the additional information acquisition unit 34 acquires site information, lesion information, treatment information, and observation mode information as additional information. Alternatively, the additional information acquisition unit 34 may acquire at least one of site information, lesion information, treatment information, and observation mode information.

In the report input screen shown in FIG. 3, when the user selects an observed organ, the display screen generation unit 46 displays an input area 120 for inputting examination results related to the selected organ on the display device 60.

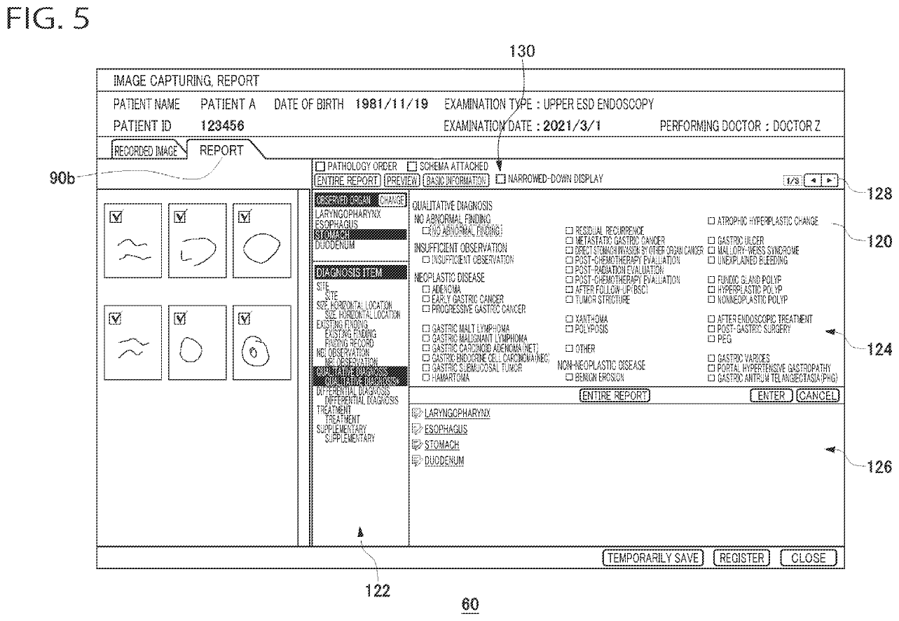

FIG. 5 shows an example of a report input screen for inputting examination results. The input area 120 includes a diagnosis target selection area 122 for selecting a diagnosis target, an examination result input area 124 for inputting examination results, and an input content display area 126 for checking the input content. The diagnosis target selection area 122 is an area for selecting a diagnosis target to be input, and when the user selects the items “observed organ” and “diagnostic item” in the diagnosis target selection area 122, an examination result input area 124 according to the selected items is displayed. In this example, “stomach” is selected as the observed organ and “qualitative diagnosis” is selected as the diagnostic item.

In the examination result input area 124, a plurality of items representing options for the examination results related to the diagnosis target are displayed. On the report input screen shown in FIG. 5, the display screen generation unit 46 reads all items that represent options for examination results of “qualitative diagnosis” of “stomach” from the item recording unit 44 and displays the items being lined up in the examination result input area 124. The user enters the examination results by checking check boxes of the corresponding items. The doctor selects each of the observed organs “pharynx”, “esophagus”, “stomach”, and “duodenum” in the diagnosis target selection area 122 and enters examination results for each observed organ in the examination result input area 124. A user interface that allows the user to input examination results by selecting items in this way can greatly reduce the time and effort required for input compared with a user interface in which the user inputs examination results in a text format.

However, the number of items included in the examination result input area 124 is very large, and the number of items is about 100 in the example shown in FIG. 5. As a result, the examination result input area 124 is formed over multiple pages (three pages in this example). Therefore, in order to select an item to be recorded in the examination report, the user needs to operate a page feeding button 128 to display a page including the item and further search for the item from the page including 30 items or more. Therefore, it takes time to perform an operation of selecting the item. Therefore, the information processor 10 is equipped with a function of narrowing down the number of items to be displayed in the examination result input area 124, and a check box 130 is provided for the user to select a narrowing down display mode on the report input screen.

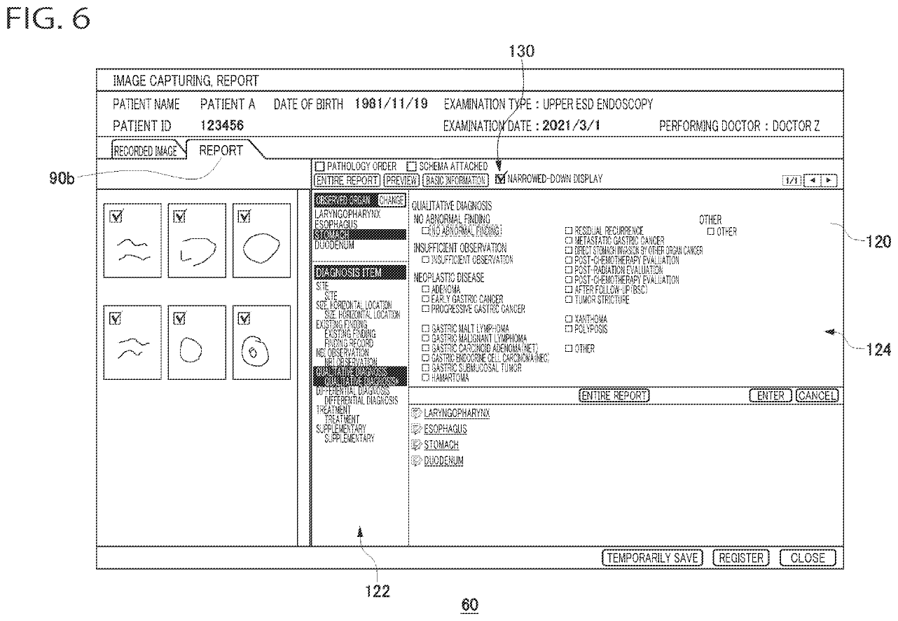

FIG. 6 shows another example of a report input screen for inputting examination results. A check mark is placed in the check box 130, which indicates that the user has performed a function of narrowing down the number of display items by the information processor 10.

As described above, the item recording unit 44 described above records a plurality of items representing options for examination results for all the observed organs and all the diagnostic items. In the narrowing down display mode, the item identification unit 42 identifies a display item group including one or more items from a plurality of items that can be included in the examination result input area 124 recorded in the item recording unit 44. By performing a narrowing down process, the item identification unit 42 reduces the number of items included in the display item group from the number of items that can be included in the examination result input area 124. The display screen generation unit 46 displays an examination result input area 124 that is for the user to input examination results related to the endoscopic image and in which one or more items included in the display item group identified by the item identification unit 42 are arranged. The user operates the input unit 62, places the mouse pointer on the check box of an item to be recorded in the report, and right-clicks, and the operation reception unit 40 thereupon receives a selection operation for the item.

The item identification unit 42 identifies a display item group based on additional information associated with an attached endoscopic image 100. By identifying the display item group based on the additional information, the item identification unit 42 can prevent items with no possibility of selection from being included in the display item group, and the display screen generation unit 46 can reduce the number of items displayed in the examination result input area 124.

In the example shown in FIG. 6, the item identification unit 42 determines items to be included in the display item group based on treatment information of an attached endoscopic image 100. The item identification unit 42 does not include items related to non-neoplastic diseases in the display item group if endoscopic treatment has been performed and includes items related to non-neoplastic diseases in the display item group if endoscopic treatment has not been performed. FIG. 6 shows the former case, in other words, items related to non-neoplastic diseases with no possibility of being selected by the user are excluded from the examination result input area 124 since the treatment information indicates that the endoscopic treatment has been performed. In FIG. 6, the number of items included in the examination result input area 124 is 23, which is significantly reduced as compared with the number of items included in the examination result input area 124 shown in FIG. 4.

The item identification unit 42 may identify the display item group based on at least one of the observation mode information, the site information, and the lesion information. For example, the item identification unit 42 excludes an “NBI observation” item in the diagnostic item from the display item group if the observation mode information does not include NBI when a plurality of items that can be included in the diagnostic target selection area 122 are recorded in the item recording unit 44. Therefore, the display screen generation unit 46 displays a diagnostic item group that does not include the NBI observation item in the diagnosis target selection area 122. As described, the item identification unit 42 may identify a display item group in which the number of items has been narrowed down based on additional information.

The examination result accumulation unit 5 records past examination results of a plurality of patients. The examination results of a plurality of patients accumulated in the examination result accumulation unit 5 are not limited to those acquired at one medical facility but may include those acquired at a plurality of medical facilities. The item identification unit 42 may identify a display item group in which the number of items is narrowed down based on the past examination results of the plurality of patients recorded in the examination result accumulation unit 5. For example, the item identification unit 42 may extract items that satisfy a predetermined display condition from past examination results of the plurality of patients recorded in the examination result accumulation unit 5 and then include the extracted items in the display item group. The examination result acquisition unit 36 reads and acquires the past examination results of the plurality of patients from the examination result accumulation unit 5.

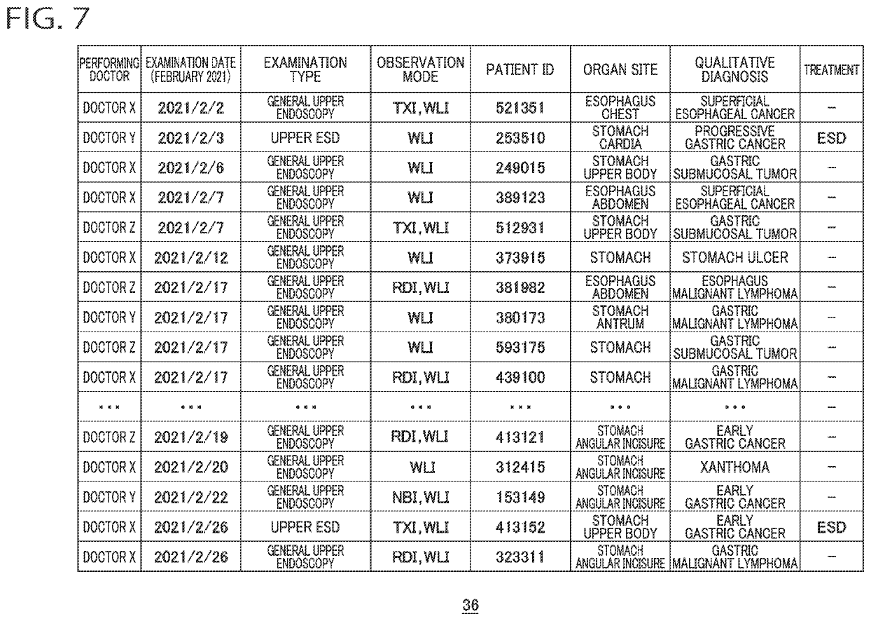

FIG. 7 shows examples of the past examination results of the plurality of patients. As past examination results, the examination result accumulation unit 5 records examination information including: information indicating the observation mode; information indicating a diagnosed site; information on a diagnosed lesion; and information on performed endoscopic treatment. The examination result accumulation unit 5 may record examination information including at least one of the information indicating the observation mode, the information indicating a diagnosed site, the information on a diagnosed lesion, and the information on performed endoscopic treatment. In this example, the examination result acquisition unit 36 reads examination results for a disease diagnosed in February 2021 from the examination result accumulation unit 5. In the examination result accumulation unit 5, examination results of patients who have not been diagnosed with a disease (that is, no abnormal findings) are also accumulated, and examination results before February 2021 are also accumulated.

The item identification unit 42 sets display conditions that include information common to past examination information and additional information associated with the attached endoscopic images 100. Referring to FIG. 4, the additional information associated with the attached endoscopic images 100 is as follows:
Observation Mode Information WLI, NBI
Site Information Stomach
Lesion Information Available
Treatment Information Available

The item identification unit 42 sets an observation mode of either “WLI” or “NBI”, the organ with a diagnosed disease being “stomach”, and the same qualitative diagnosis having been made three or more times in one month as display conditions, and then searches for an item, a disease name, that satisfies the display conditions. These display conditions mean that in an examination using at least one of the observation modes of “WLI” or “NBI”, a search is made for items, disease names, diagnoses for “stomach” three or more times in one month. Under these display conditions, the count is not limited to three times as long as the count is set for making a search for items, which are disease names, that are frequently diagnosed for “stomach” when the examination is performed using “WLI” or “NBI”, and the count may be set for each hospital facility.

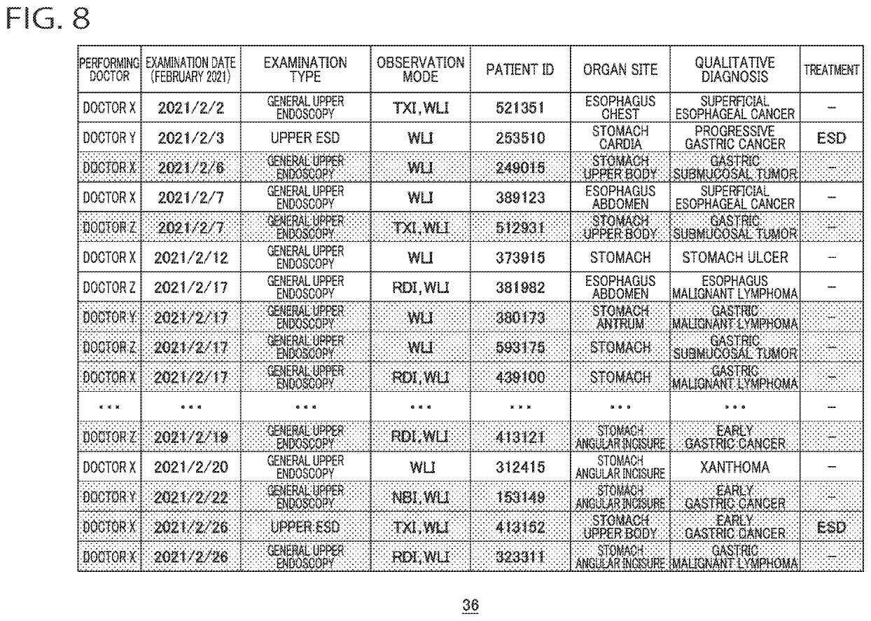

FIG. 8 shows an example of a search result for examination results that satisfy the display conditions. Based on this search result, it can be found that the items, disease names, diagnosed for “stomach” three or more times in one month in an examination using at least one of the observation modes of “WLI” and “NBI” are “early gastric cancer”, “gastric malignant lymphoma”, and “gastric submucosal tumor”. In other words, in this medical facility, an examination using at least one of the observation modes of “WLI” and “NBI” leads to a high probability of diagnosing “early gastric cancer”, “gastric malignant lymphoma”, and “gastric submucosal tumor”. Therefore, the item identification unit 42 may identify items that satisfy the display conditions, that is, a display item group including “early gastric cancer”, “gastric malignant lymphoma”, and “gastric submucosal tumor” from among a plurality of items that can be displayed in the examination result input area 124. The item identification unit 42 does not include other items for neoplastic diseases and items for non-neoplastic diseases in the display item group. The item identification unit 42 may search for items that satisfy these display conditions for a predetermined period, for example, the past year.

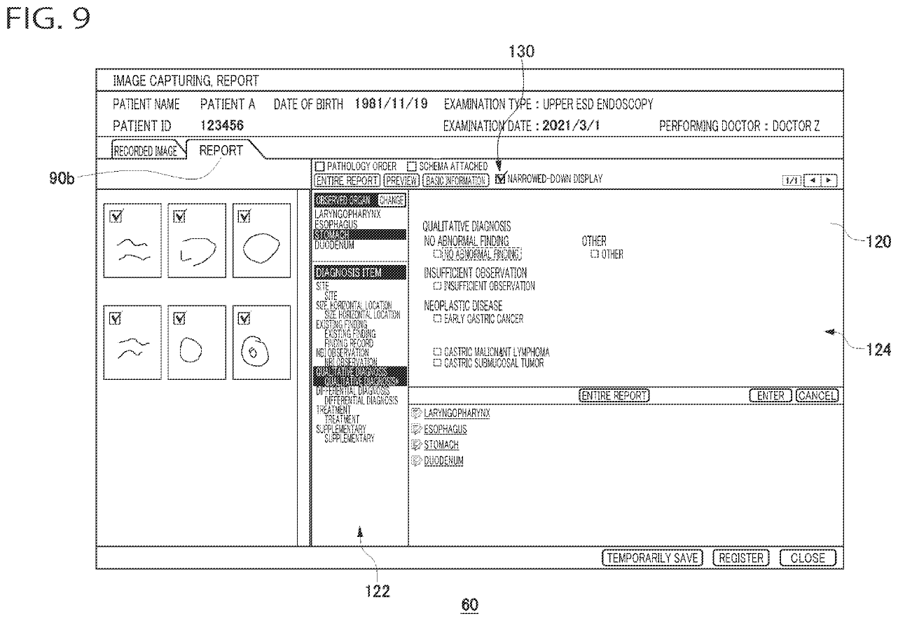

FIG. 9 shows another example of the report input screen for inputting examination results. The display screen generation unit 46 displays an examination result input area 124 in which one or more items included in the display item group identified by the item identification unit 42 are arranged on the display device 60. Items related to neoplastic diseases and items for non-neoplastic diseases are not displayed except for “early gastric cancer”, “gastric malignant lymphoma”, and “gastric submucosal tumor”, which satisfy the display conditions. Thus, the user can efficiently select these items. If the current diagnosis is not any of “early gastric cancer”, “gastric malignant lymphoma”, and “gastric submucosal tumor”, the user only needs to uncheck the check box 130 so as to exit the narrowing down display mode. In that case, as shown in FIG. 5, all items end up being displayed in the examination result input area 124.

The information processor 10 according to the embodiment also has an automatic check function for display items in addition to the function of narrowing down display items. This automatic check function causes the display screen generation unit 46 to display one or more items that have not been selected by an operation from the user in a manner indicating that the items are already selected. Therefore, when the user opens the report input screen, the one or more items may be displayed as checked. Check marks entered by the automatic check function are preferably displayed in a manner different from those provided by a user operation performed on check boxes, in order to allow the user to recognize the check marks as those entered by the automatic check function.

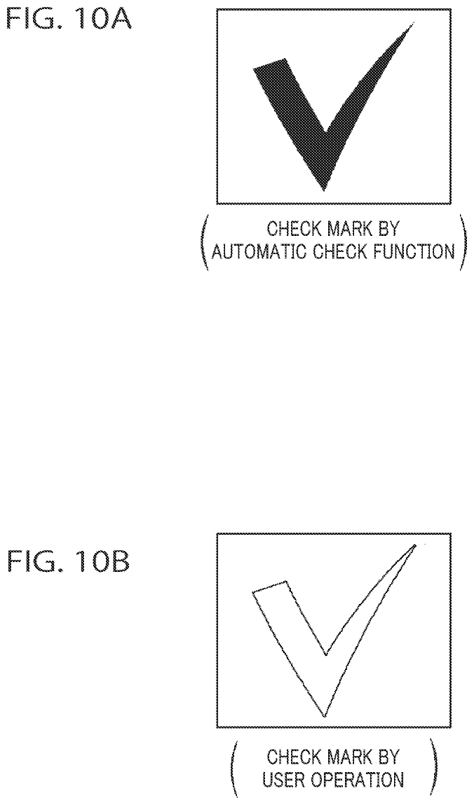

FIG. 10A shows an example of a check mark provided by the automatic check function, and FIG. 10B shows an example of a check mark provided by a user operation. As described, the display screen generation unit 46 makes a first display mode indicating that an item not selected by an operation from the user has been selected and a second display mode indicating that an item selected by an operation from the user has been selected different from each other. This allows the user to distinguish between items he or she has selected and items selected by the automatic check function.

The automatic check unit 50 performs the automatic check function. From the examination result accumulation unit 5, the examination result acquisition unit 36 reads and acquires examination result obtained when the patient, who has undergone the examination subject to the report, underwent the same type of examination in the past. As described in the upper part of the report input screen, the patient ID of the patient for the examination subject to the report is “123456”, and the examination type is “upper ESD endoscopy”. The examination result acquisition unit 36 acquires examination results obtained when Patient A underwent upper ESD endoscopy in the past from the examination result accumulation unit 5.

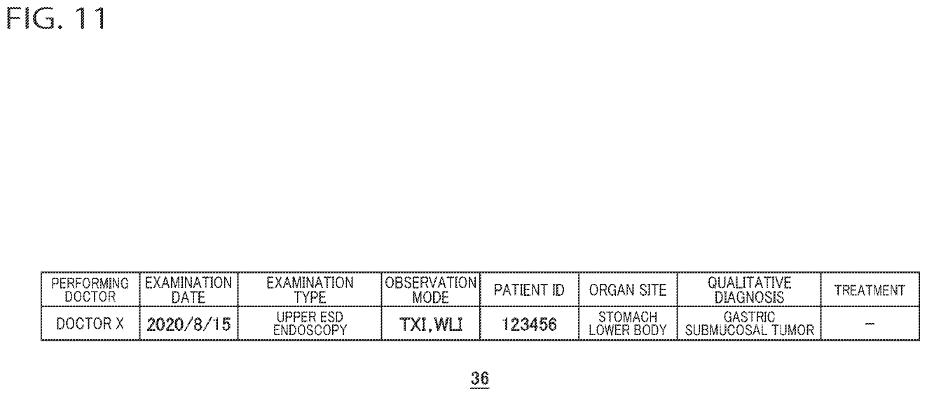

FIG. 11 shows an example of past examination results of Patient A. Patient A underwent an upper ESD endoscopy on Aug. 15, 2020 and was diagnosed with a “gastric submucosal tumor” in the “stomach, lower body”. Therefore, even in this upper ESD endoscopy, there is a high possibility that “gastric submucosal tumor” will be diagnosed, and the automatic check unit 50 notifies the display screen generation unit 46 to automatically check the item “gastric submucosal tumor”.

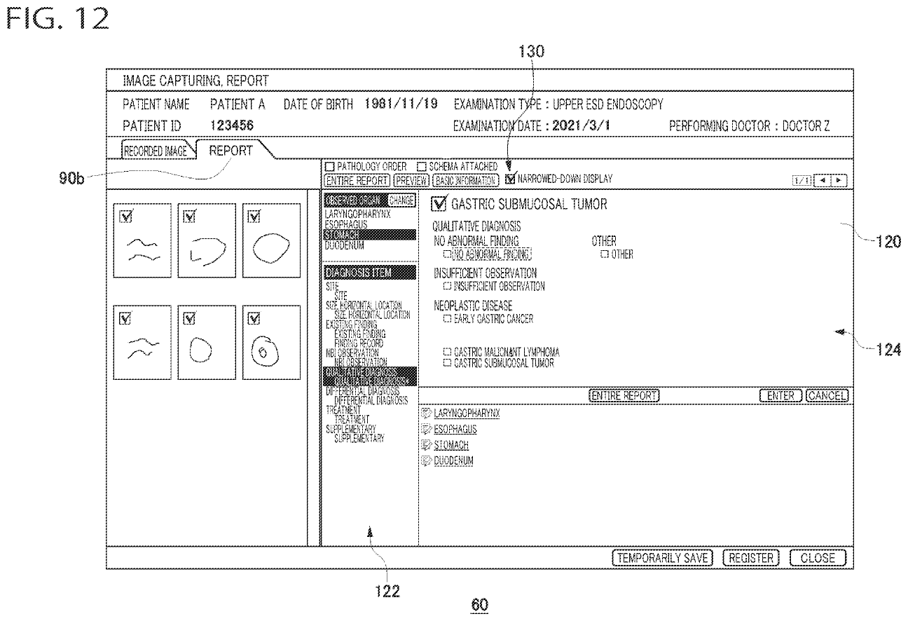

FIG. 12 shows another example of a report input screen for inputting examination results. The display screen generation unit 46 displays an examination result input area 124 in which one or more items included in the display item group identified by the item identification unit 42 are arranged. At this time, the display screen generation unit 46 displays the items as notified to be automatically checked by the automatic check function in the examination result input area 124 in a manner indicating that the items are already selected.

On the report input screen shown in FIG. 12, the item “gastric submucosal tumor” is placed at the upper position of the examination result input area 124, and the check box is displayed as checked. As described, the display screen generation unit 46 may display items corresponding to examination results for the same type of examination performed in the past in a manner indicating that the items are already selected. Thereby, the user does not have to perform an operation of selecting the items, and the efficiency of the input work can be improved. When deselecting the items, the user just needs to place the mouse pointer on the check box of each item and right-click.

The display screen generation unit 46 arranges items to be displayed in a manner indicating that the items are already selected above items to be displayed in a manner indicating that the items are not being selected. The selected “gastric submucosal tumor” is arranged at the highest position on the report input screen shown in FIG. 12, which allows the user to recognize the automatically checked item without fail. In the examination result input area 124 shown in FIG. 12, the same “gastric submucosal tumor” is displayed in a selectable manner below the selected “gastric mucosal tumor” displayed at the highest position. In the case of such duplication, the display screen generation unit 46 may hide the one of the items that has not been checked.

The automatic check unit 50 may determine items to be automatically checked based on the site information associated with an attached endoscopic image 100. In the embodiment, the additional information acquisition unit 34 acquires the site information “stomach, lower body” as the site where a lesion exists, and the automatic check unit 50 therefore notifies the display screen generation unit 46 that an item “lower body” of the stomach is automatically checked.

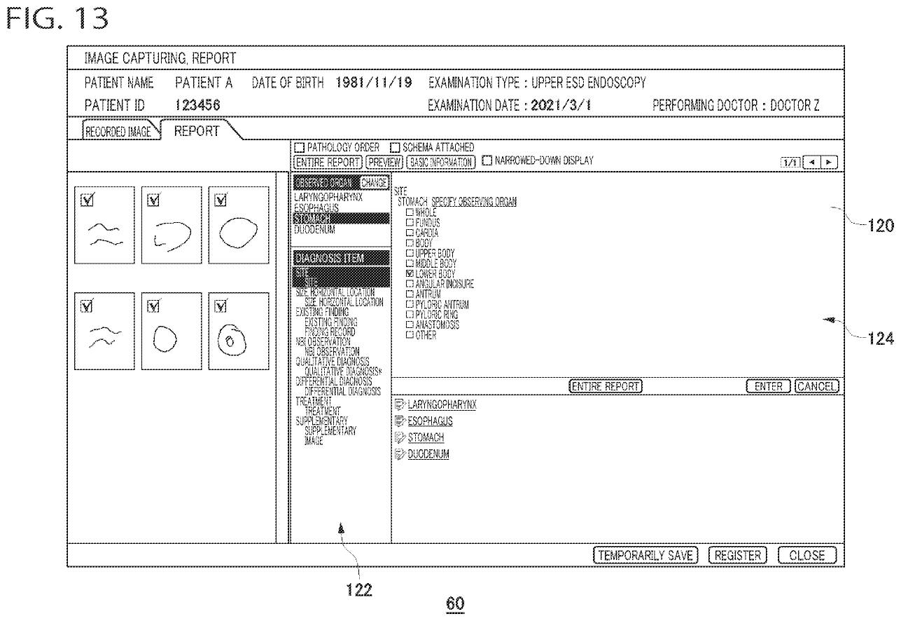

FIG. 13 shows another example of the report input screen for inputting examination results. The narrowing down display mode is not set on this report input screen, and the display screen generation unit 46 displays an examination result input area 124 in which all items associated with an observed organ “stomach” and a diagnostic item “site” are arranged. The display screen generation unit 46 displays “lower body”, which is an item corresponding to the site information, on the display device 60 in a manner indicating that the item has been selected. Thereby, the user does not have to perform an operation of selecting “lower body”, and the efficiency of the input work can be improved.

The medical care assistance system 1 may be configured to manage implementation items for calculating the cost of examination. After the completion of the endoscopy, the doctor or nurse inputs implementation items for a medical procedure performed in the examination into the medical care assistance system 1. The information processor 10 converts the implementation items of the medical care assistance system 1 into items on the input screen for the report.

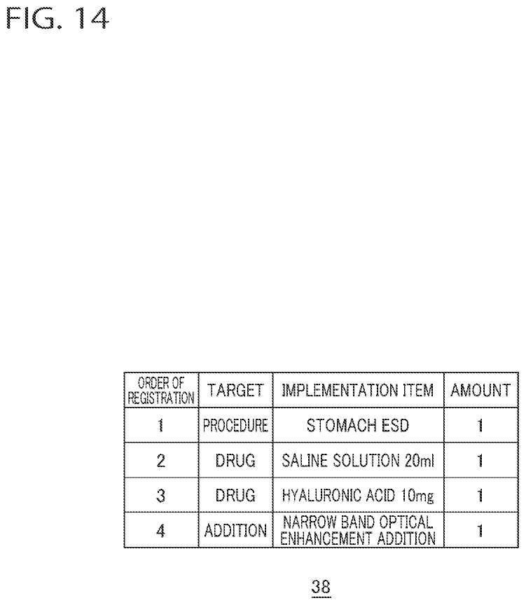

FIG. 14 shows examples of medical procedure implementation items input to the medical care assistance system 1 for the upper ESD endoscopy of Patient A. The implementation information acquisition unit 38 receives user input and acquires implementation items for the upper ESD endoscopy. The implementation items for a medical procedure are generated according to a master table of implementation items, and it is thus necessary to convert the implementation items into items used on a report input screen of the medical care assistance system 1.

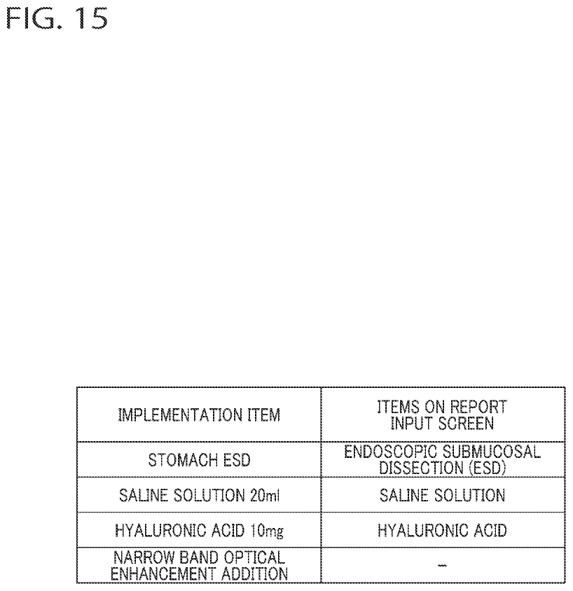

FIG. 15 shows a table for converting implementation items in the medical care assistance system 1 into items on a report input screen in the medical care assistance system 1. The automatic check unit 50 converts an implementation item “gastric ESD” into an item “endoscopic submucosal dissection (ESD)”, an implementation item “saline solution 20 ml” into an item “saline solution”, and an implementation item “hyaluronic acid 10 mg” into an item “hyaluronic acid”. No item for conversion is associated with an implementation item “narrow band optical enhancement addition”, and the automatic check unit 50 thus does not convert the implementation item “narrow band optical enhancement addition”. The automatic check unit 50 determines items to be automatically checked from the converted items. The automatic check unit 50 notifies the display screen generation unit 46 that the automatic check unit 50 automatically checks the items “endoscopic submucosal dissection (ESD)”, “saline solution”, and “hyaluronic acid”.

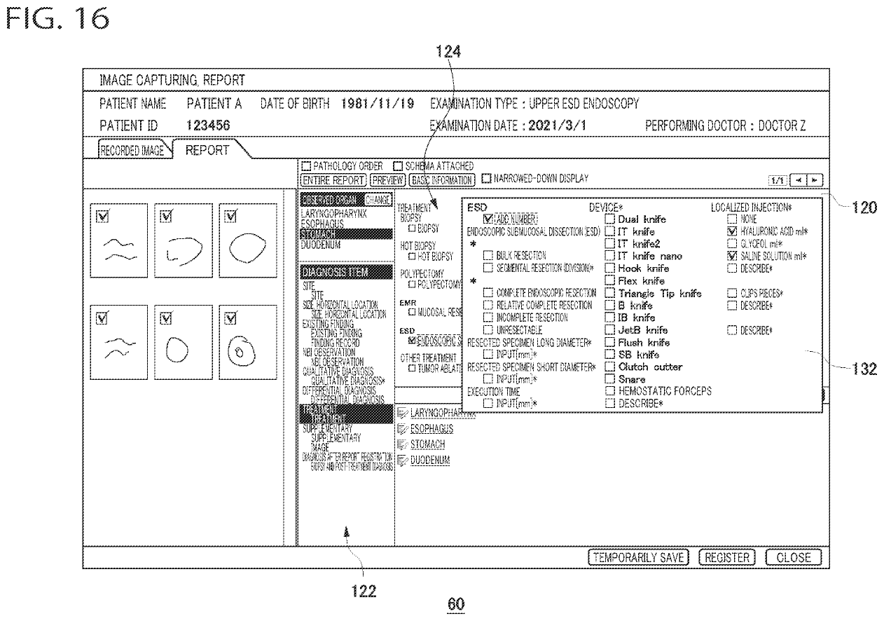

FIG. 16 shows another example of the report input screen for inputting examination results. The narrowing down display mode is not set on this report input screen, and the display screen generation unit 46 displays an examination result input area 124 in which all items associated with an observed organ “stomach” and a diagnostic item “treatment” are arranged. The display screen generation unit 46 displays “ESD”, “saline solution”, and “hyaluronic acid”, which are items corresponding to the implementation information for a medical procedure, on the display device 60 in a manner indicating that the items are already selected. “Saline solution” and “hyaluronic acid” are included in a detailed input screen 132, and the detailed input screen 132 is displayed when the “ESD” item in the examination result input area 124 is selected. As described, the display screen generation unit 46 displays check boxes for items corresponding to implementation information for a medical procedure as checked; thereby, the user does not have to perform an operation of selecting these items, and the efficiency of the input work can be improved.

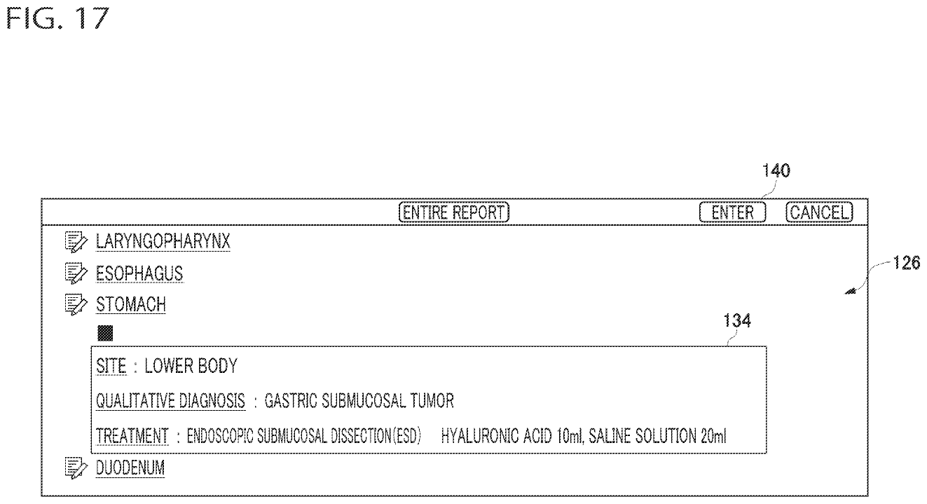

FIG. 17 shows a display example of the input content display area 126. The display screen generation unit 46 displays examination results that are being input in the input content display area 126. The display screen generation unit 46 displays item information on selected items in the input content display area 126 when the user selects one organ, a stomach, and inputs the first finding on the report input screen. In the example shown in FIG. 17, the display screen generation unit 46 displays finding information 134 that is being input, that is, information on the items selected in the examination result input area 124. The finding information 134 that is being input may be displayed in a manner indicating that the finding information 134 is being input, for example, while being enclosed with a frame since the finding information 134 has not been determined to be final. When the user operates an enter button 140 in this state, the input finding information 134 is determined to be final.

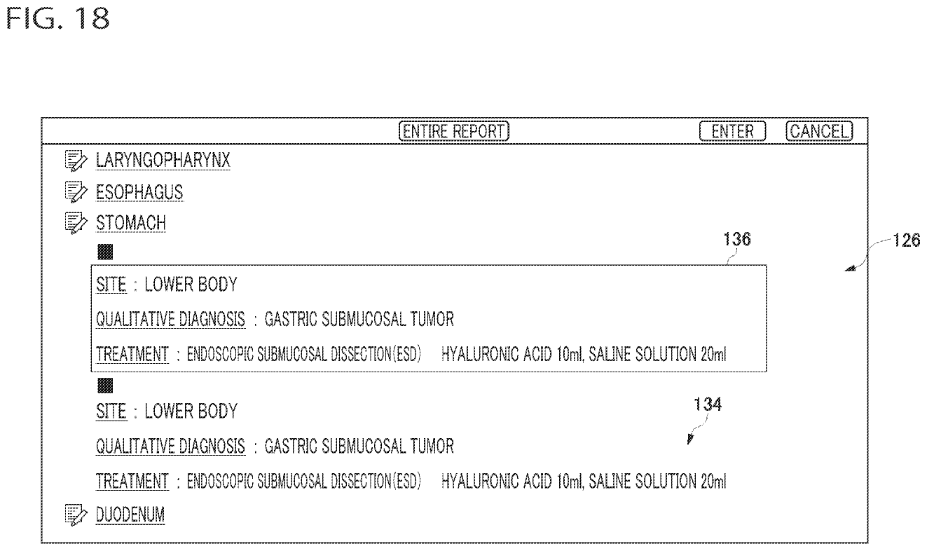

FIG. 18 shows a display example of the input content display area 126. The display screen generation unit 46 may display at least a part of item information on selected items when the user selects the same organ, a stomach, and inputs the second finding on the report input screen. In the example shown in FIG. 18, the display screen generation unit 46 displays the same content as the finding information 134 as the finding information 136 that is being input. Alternatively, the display screen generation unit 46 may display only a part of the finding information 134. Even in this case, the display screen generation unit 46 displays at least a part of the item information on selected items, and the user can thereby omit the trouble of entering duplicate content.

By using the function of narrowing down display items and the automatic check function for display items by the information processor 10, the user can shorten the report input time. The user selects items other than the items selected by the automatic check function by operating the input unit 62 and inputs examination results. When the user inputs all the examination results, he or she operates a register button to confirm the input content. The examination results that have been input are transmitted to the examination result accumulation unit 5, and the report input work is completed.

Described above is an explanation on the present disclosure based on the embodiments. These embodiments are intended to be illustrative only, and it will be obvious to those skilled in the art that various modifications to constituting elements and processes could be developed and that such modifications are also within the scope of the present disclosure. In the embodiments, endoscopic images are shown as examples of medical images. The function of narrowing down display items and the automatic check function can be used not only for endoscopic images but also for other types of medical images.

What is claimed is:

1. A medical care assistance system comprising:
one or more processors comprising hardware, wherein the one or more processors are configured to:
acquire a medical image;
acquire additional information associated with the medical image; and
generate an input screen that is for a user to enter an examination result on the medical image and in which one or more items included in a display item group that is identified based on the additional information are arranged; and
receive a selection operation for an item displayed on the input screen.

2. The medical care assistance system according to claim 1, wherein the one or more processors are configured to:
determine whether there is a possibility for a user to select an item based on the additional information; and
not display an item with no possibility of being selected.

3. The medical care assistance system according to claim 1, wherein the one or more processors are configured to:
identify the display item group to be displayed based on the additional information from a plurality of items that can be included in the input screen.

4. The medical care assistance system according to claim 2, wherein the one or more processors are configured to:
acquire information indicating whether treatment with an endoscopic treatment tool has been performed as the additional information; and
not include items related to nonneoplastic diseases in the display item group when endoscopic treatment has been performed and include items related to nonneoplastic diseases in the display item group when endoscopic treatment has not been performed.

5. The medical care assistance system according to claim 2, wherein the one or more processors are configured to:
acquire at least one of information indicating an observation mode, information indicating a site of a subject, and information on a lesion as the additional information; and
identify the display item group based on at least one of the information indicating the observation mode, the information indicating the site of the subject, and the information on the lesion.

6. The medical care assistance system according to claim 2, further comprising:
a recording device that records past examination results of a plurality of patients,
wherein the one or more processors are configured to:
identify the display item group based on the past examination results of the plurality of patients recorded in the recording device.

7. The medical care assistance system according to claim 6, wherein the one or more processors are configured to:
extract items that satisfy a predetermined display condition from the past examination results of the plurality of patients recorded in the recording device and then include the extracted items in the display item group.

8. The medical care assistance system according to claim 7,
wherein, as the past examination results, the recording device records examination information including at least one of information indicating an observation mode, information indicating a diagnosed site, information on a diagnosed lesion, and information on performed endoscopic treatment, and
wherein the one or more processors are configured to:
acquire at least one of information indicating the observation mode, information indicating a site of a subject, information on a lesion, and information on endoscopic treatment as the additional information associated with the medical image; and
include items that satisfy the predetermined display condition including information common to the examination information and the additional information in the display item group.

9. The medical care assistance system according to claim 2, wherein the one or more processors are configured to:
display one or more items that have not been selected by an operation from the user in a manner indicating that the items are already selected.

10. The medical care assistance system according to claim 9, wherein the one or more processors are configured to:
make a first display mode indicating that an item not selected by an operation from the user has been selected different from a second display mode indicating that an item selected by an operation from the user has been selected.

11. The medical care assistance system according to claim 9, wherein the one or more processors are configured to:
arrange the one or more items to be displayed in a manner indicating that the items are already selected above items to be displayed in a manner indicating that the items are not being selected.

12. The medical care assistance system according to claim 9, wherein the one or more processors are configured to:
acquire information indicating a site of a subject as the additional information associated with the medical image; and
display an item corresponding to the site information of the subject in a manner indicating that the item is already selected.

13. The medical care assistance system according to claim 9, wherein the one or more processors are configured to:
acquire an examination result for a patient who has undergone examination obtained when the patient underwent the same type of examination in the past; and
display an item corresponding to the examination result in the past in a manner indicating that the item is already selected.

14. The medical care assistance system according to claim 9, wherein the one or more processors are configured to:
acquire implementation information for a medical procedure in examination; and
display an item corresponding to the implementation information for the medical procedure in a manner indicating that the item is already selected.

15. The medical care assistance system according to claim 9, wherein the one or more processors are configured to:
display item information on selected items when the user selects one organ and enters a finding on an input screen and display at least a part of the item information on selected items when the user selects the same organ again and enters a different finding.

16. A medical care assistance system comprising:
one or more processors comprising hardware, wherein the one or more processors are configured to:
capture a medical image;
acquire the medical image;
acquire additional information associated with the medical image;
generate an input screen that is for a user to enter an examination result on the medical image and in which one or more items included in a display item group identified based on the additional information are arranged; and
receive a selection operation for an item displayed on the input screen.

17. The medical care assistance system according to claim 16, wherein the one or more processors are configured to:
associate at least one of information indicating an observation mode, information indicating a site of a subject, information on a lesion, and information on endoscopic treatment with the medical image as the additional information; and
identify the display item group based on the additional information.

18. An input assistance method for medical care information, comprising:
acquiring a medical image;
acquiring additional information associated with the medical image;
generating an input screen that is for a user to enter an examination result on the medical image and in which one or more items included in a display item group identified based on the additional information are arranged; and
receiving a selection operation for an item displayed on the input screen.
